(19) United States
(12) Patent Application Publication
(10) Pub. No.: US 2008/0102496 A1
(43) Pub. Date: May 1, 2008

(54) Expression of Nitrile Hydratases in a Two-Vector Expression System

(75) Inventors: Stefan Verseck, Hanau (DE); Steffen Osswald, Rodenbach (DE); Wai-Yee Phong, Singapore (SG); Klaus Liebeton, Zwingenberg (DE); Jurgen Eck, Bensheim (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: BRAIN BIOTECHNOLOGY RESEARCH AND INFORMATION NETWORK AG, Zwingenberg (DE)

(21) Appl. No.: 10/593,362

(22) PCT Filed: Feb. 18, 2005
(86) PCT No.: PCT/EP05/01688

§ 371 Date: Sep. 18, 2006

(30) Foreign Application Priority Data
Mar. 20, 2004 (DE) 10 2004 031 843.5

(51) Int. Cl. C12N 1/20; C12N 15/00; C12N 15/64; C12N 9/88; C12P 7/40

(52) U.S. Cl. 435/91.4; 435/136; 435/232; 435/252.8; 435/320.1

(57) ABSTRACT

The present invention relates to a novel system for expressing nitrile hydratase. In this system, the nitrile hydratases, which are composed of subunits, are formed such that the respective subunits are located on different plasmids and are expressed simultaneously in E. coli.

The present invention relates to an expression system for preparing nitrile hydratases. Nitrile hydratases consist of different subunits. The present system makes it possible to prepare nitrile hydratases in a manner superior to that of the prior art by expressing the nucleic acid sequences encoding these subunits separately on separate plasmids.

The amide and carboxylic acid structural classes are becoming increasingly important as precursors of fine chemicals. Special aminoamides and (proteinogenic and nonproteinogenic) amino acids are key intermediates for synthesizing pharmaceutical and agrochemical products as well as in the foodstuff field. Enantiomerically pure amides and amino acids, in particular, play a role which is becoming ever more important in the abovementioned application fields.

Aminonitrile precursors, as are required for preparing the abovementioned compound classes, can readily be obtained in racemic form using the Strecker synthesis. The nitrites which are obtained in this way can then be converted by means of chemical or enzymic hydrolysis into the corresponding amides and carboxylic acids.

Three enzymes which can participate in the enzymic hydrolysis of nitrites are known. Nitrilases convert a nitrile function directly into the acid, whereas nitrile hydratases (E.C. 4.2.1.84) form the corresponding amide under these circumstances. This latter can finally be converted into the corresponding carboxylic acid by an amidase (E.C. 3.5.1.4) (scheme 1).

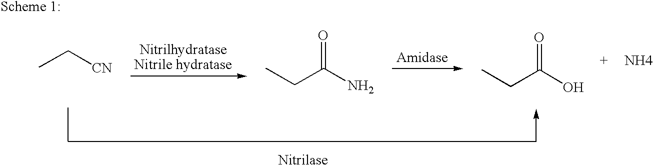

Hydrolyzing nitrites to the corresponding amides and acids using isolated enzymes or whole-cell catalysts helps to save large quantities of salt which would otherwise accrue in connection with the neutralization step following the chemical hydrolysis of nitrites. For this reason, the enzymic hydrolysis of nitrites to, for example, aminoamides and/or amino acids constitutes a more sustainable production process.

In their active form, nitrile hydratases consist of 2 nonhomologous α- and β-subunits. These subunits form heterodimers and tetramers, while decamers have even been demonstrated in the case of Rhodococcus rhodochrous J1. While the α- and β-subunits are of approximately the same size, they are otherwise in no way similar to each other. Nitrile hydratases are metalloproteins which contain Fe³⁺ or Co³⁺ (Bunch A. W. (1998), Nitriles, in: Biotechnology Volume 8a, Biotransformations I, Chapter 6, Eds.: Rehm H J, Reed G, Wiley-VCH, p. 277-324; Shearer J, Kung I Y, Lovell S, Kaminsky W, Kovacs J A (2001) Why is there a “inert” metal center in the active site of nitrile hydratase? Reactivity and ligand dissociation from a five-coordinate Co(III) nitrile hydratase model. J Am Chem Soc 123: 463-468; Kobayashi M, Shimizu S (2000) Nitrile hydrolases. Current Opinion in Chemical Biology 4: 95-102).

One of the greatest challenges to date has been that of heterologously preparing nitrile hydratases in a suitable host, preferably in E. coli. This Gram-negative bacterium is known for its ability to express heterologous proteins at high rates. Another advantage is the yield of biomass in high-cell-density fermentations using E. coli. In this connection, it is possible to achieve productivities of more than 100 g of dry biomass (DBM) in from 24 to 44 hours (Lee SY (1996) High cell-density culture of Escherichia coli. TIBTECH 14:98-105; Riesenberg D, Guthke R (1999) High-cell-density cultivation of microorganisms. Appl Microbiol Biotechnol 51:422-430).

Most of the sequences for the nitrile hydratase α- and β-subunits are known from the genus Rhodococcus. However, it is precisely this genus whose nitrile hydratases have thus far only been expressed in E. coli with particular difficulty (Ikehata O, Nishiyama M, Horinouchi S, Beppu T (1989) Primary structure of nitrile hydratase deduced from the nucleotide sequence of a Rhodococcus species and its expression in Escherichia coli. Eur J Biochem 181: 563-570).

The literature describes one-vector systems for expressing nitrile hydratases, the specific activities of which systems lie between 4.2 and 12.2 U/mg of total protein in the case of codependent R. rhodochrous J1 nitrile hydratases (Kobayasjhi M, Nishiyama M, Nagasawa T, Horinouchi S, Beppu T, Yamada H (1991) Cloning, nucleotide sequence and expression in Escherichia coli of two cobalt-containing nitrole hydratase genes from Rhodococcus rhodochrous. Biochim Biophys Acta 1129: 23-33) and 452 U/mg of total protein in the case of an iron-dependent Rhodococcus spec. N-771 nitrile hydratase (Njori M, Yohda M, Odaka M, Matsushita Y, Tsujimura M, Yoshida T, Dohmae N, Takio K Endo I (1999) Functunal expression of Nitrile hydratases in E. coli: Requirement of a nitrile hydratase activator and a post-translational modification of a ligand cysteine. J Biochem 125: 696-704), which corresponds roughly to approx. 248 U/mg of DBM (dry biomass) (calculation in accordance with Goodsell DS (1991) Inside a cell. TIBS 16: 203-206). Interestingly, it was not possible to reproduce the latter activity with nitrile hydratases from R. erythropolis, which is closely related to Rhodococcus spec. N-711, using similar vector systems and dispositions of the structural genes. There was, therefore, still a need for processes and systems which permit the enzymes under consideration to be made available in a manner which is adequate for industrial scale preparations.

The skilled person is already familiar with the use of two-vector expression systems for heterologously expressing recombinant proteins in E. coli, for example for forming the motor protein kinesin (Skowronek K, Kasprzak A (2002) A two-plasmid system for independent genetic manipulation of subunits of homodimeric proteins and selective isolation of chimeric dimers. Analytical Biochemistry 300: 185-191), the plasminogen proactivator streptokinase (Yazdani SS, Mukherjee K J (2002) Continuous-culture studies on the stability and expression of recombinant streptokinase in Escherichia coli; stability and expression of streptokinase in continuous culture. Bioprocess and Biosystems Engineering 24(6): 341-346), the complex of two human proteins (hematopoietic cell tyrosine phosphatase and mitogen protein kinase; Kholod N, Mustelin T (2001) Novel vectors for co-expression of two proteins in E. coli. 31: 322-328) or the human CKMB creatine kinase (WO95/12662).

However, no heteromeric enzymes, such as the nitrile hydratases, which are used as biocatalysts in the chemical industry have thus far been expressed using such a system.

The object was, therefore, to develop an expression system which makes it possible to efficiently express both cobalt-dependent and iron-dependent nitrile hydratases actively in E. coli. In particular, the system according to the invention should be able to make the enzymes under consideration available at a rate of expression which is higher, and, where appropriate, in forms which are more stable, than in the case of the prior art in order, in this way, to make the use of these enzymes on an industrial scale advantageous from ecological and economic points of view.

These objects, and other objects which are not specified in detail but which ensue in an obvious manner from the prior art, are achieved by specifying an expression system having the features of the present claim 1. Claims 2 to 8 relate to preferred embodiments of the expression system according to the invention. Claims 9 and 10 are directed towards processes for preparing nitrile hydratases and/or (amino)carboxylic acids or (amino)carboxamides. Claim 11 protects a host organism which is equipped with the expression system.

The set object is achieved, extremely advantageously and nonetheless entirely surprisingly, by, in the case of an expression system for simultaneously expressing the nucleic acid sequences encoding the different subunits of a nitrile hydratase, the expression system possessing in each case at least one plasmid containing at least one nucleic acid sequence encoding the respective subunit. Using the proposed expression system, it is possible to heterologously express the nucleic acid sequences under consideration in a manner which is adequate for industrial scale preparations. In this connection, it may be particularly surprising that simply expressing the nucleic acid sequences encoding the corresponding nitrile hydratase subunits, which nucleic acid sequences are in fact organized in one operon, separately on different plasmids contributes to increasing the activity of the resulting nitrile hydratases by a factor of >8 as compared with the “normal” expression. It was not possible to deduce this in an obvious manner from the prior art.

The expression system according to the invention can be used in all the host organisms which the skilled person takes into consideration for the present purpose. Microorganisms which are to be mentioned in this regard are organisms such as yeast, such as Hansenula polymorpha, Pichia sp. and Saccharomyces cerevisiae, prokaryotes, such as E. coli and Bacillus subtilis, or eukaryotes, such as mammalian cells, insect cells or plant cells. Host organisms into which plasmids containing the nucleic acid sequences can be cloned are used for replicating and isolating a sufficient quantity of the recombinant enzyme. The methods used for this purpose are well known to the skilled person (Sambrook, J.; Fritsch, E. F. and Maniatis, T. (1989), Molecular cloning: a laboratory manual, 2nd ed., Cold Spring Harbor Laboratory Press, New York). Preference is given to using E. coli strains for this purpose. The following are very particularly preferred: E. coli XL1 Blue, NM 522, JM101, JM109, JM105, RR1, DH5α, TOP 10-, HB101, BL21 codon plus, BL21 (DE3) codon plus, BL21, BL21 (DE3) and MM294. Plasmids which are preferably used to clone the gene construct containing the nucleic acid according to the invention into the host organism are likewise known to the skilled person (see also PCT/EP03/07148; see below). Very particular preference is given to an expression system which is present in E. coli BL21 as the host.

Promoters are DNA sequence regions from which transcription of a gene or operon is controlled. The promoters which are particularly advantageous for implementing the invention and which are to be used, in particular, in E. coli are known to the skilled person (Sambrook, J.; Fritsch, E. F. and Maniatis, T. (1989), Molecular cloning: a laboratory manual, 2nd ed., Cold Spring Harbor Laboratory Press, New York). It has now proved to be advantageous if the expression of the nucleic acid sequences encoding the subunits is in each case under the control of the same promoter so that the nucleic acid sequences encoding the subunits can be expressed at a rate which is as identical as possible. Suitable promoters can be selected from the group T7, lac, tac, trp, ara or rhamnose-inducible. Other promoters are mentioned in (Cantrell, S A (2003) Vectors for the expression of recombinant proteins in E. coli. Methods in Molecular biology 235: 257-275; Sawers, G; Jarsch, M (1996) Alternative principles for the production of recombinant proteins in Escherichia coli. Applied Microbiology and Biotechnology 46(1): 1-9). Very particular preference is given to using the T7 promoter in the expression system according to the invention (Studier, W. F.; Rosenberg A. H.; Dunn J. J.; Dubendroff J. W.; (1990), Use of the T7 RNA polymerase to direct expression of cloned genes, Methods Enzymol. 185, 61-89; or brochures supplied by the companies Novagen or Promega).

It has been found to be useful, for the ability of the expression system according to the invention to function, for particular nucleic acid sequences, which encode peptide sequences known to be helper proteins and whose functions have previously to a large extent been unknown, to be present on the corresponding plasmids. These sequences are known to the skilled person in regard to producing active nitrile hydratases (Nojiri M; Yohda M; Odaka M; Matsushita Y; Tsujimura M; Yoshida T; Dohmae N; Takio K; Endo I (1999) Functional expression of nitrile hydratase in Escherichia coli: requirement of a nitrile hydratase activator and post-translational modification of a ligand cysteine. Journal of biochemistry 125(4): 696-704). Very particular preference is given to an expression system in which at least one nucleic acid sequence encoding such a helper protein, in particular the p47K protein (Seq. ID No. 33) or the p12K protein (Seq. ID No. 31), is present per plasmid set employed. In this connection, a plasmid set denotes the plasmids which are required in accordance with the invention for constructing an active nitrile hydratase.

As explained in detail at the outset, nitrile hydratases are known from a variety of organisms (see also PCT/EP04/00338; Dissertation, see above). However, preference is given to using, in the expression system according to the invention, those nucleic acid sequences which encode nitrile hydratase subunits which have their origin in nitrile hydratases derived from Rhodococcus strains. In this connection, the nucleic acid sequences which are employed can be altered, as compared with the original sequences from Rhodococcus, by means of mutagenesis on a chemical or molecular biological basis. In this connection, particular consideration is given to those nucleic acid sequences which encode subunits which are improved, as compared with the wild-type sequences, in regard to activity and/or selectivity and/or stability. According to the invention, the improvement in the activity and/or selectivity and/or stability denotes that the enzymes under consideration are more active and/or more selective or less selective, or more stable under the reaction conditions employed. While the activity and the stability of the enzymes should naturally, for the industrial application, be as high as possible, the selectivity is considered to be improved when either the substrate selectivity decreases but the enantioselectivity of the enzymes is increased.

The skilled person is sufficiently familiar with the procedure for using mutagenesis methods to improve the nucleic acid sequences according to the invention or the polypeptides which they encode. The mutagenesis methods which are suitable are any methods which are available to the skilled person for this purpose. In particular, these methods are saturation mutagenesis, random mutagenesis, in-vitro recombination methods and site-directed mutagenesis (Eigen, M. and Gardiner, W. (1984), Evolutionary molecular engineering based on RNA replication, Pure Appl. Chem. 56, 967-978; Chen, K. and Arnold, F. (1991), Enzyme engineering for nonaqueous solvents: random mutagenesis to enhance activity of subtilisin E in polar organic media. Bio/Technology 9, 1073-1077; Horwitz, M. and Loeb, L. (1986), Promoters Selected From Random DNA-Sequences, Proc Natl Acad Sci USA 83, 7405-7409; Dube, D. and L. Loeb (1989), Mutants Generated By The Insertion of Random Oligonucleotides Into The Active-Site of The Beta-Lactamase Gene, Biochemistry 28, 5703-5707; Stemmer, P. C. (1994), Rapid evolution of a protein in vitro by DNA shuffling, Nature 370, 389-391 and Stemmer, P. C. (1994), DNA shuffling by random fragmentation and reassembly: In vitro recombination for molecular evolution. Proc Natl Acad Sci USA 91, 10747-10751).

Particular preference is given to the nucleic acid sequences encoding the nitrile hydratase subunits being derived from Rhodococcus strains, in particular R. erythropolis 870-AN019.

In another preferred embodiment, the nucleic acid sequences employed are altered such that they correspond particularly well to the E. coli codon usage. It has been found that the yields of the enzymes obtained can be increased still further in proportion to the extent to which the codon usage of the gene to be expressed corresponds to that of E. coli. Particular preference is therefore given to modifying the nucleic acid sequences which encode the nitrile hydratase subunits in conformity with the E. coli codon usage. “Codon usage” is understood as being the fact that different organisms use different base triplets, which encode the same amino acids (degeneracy of the genetic code) to differing extents.

In principle, the plasmids or vectors which can be used are any types which are available to the skilled person for this purpose. These plasmids and vectors are listed, for example, in Studier and coworkers (Studier, W. F.; Rosenberg A. H.; Dunn J. J.; Dubendroff J. W.; (1990), Use of the T7 RNA polymerase to direct expression of cloned genes, Methods Enzymol. 185, 61-89) or the brochures supplied by the companies Novagen, Promega, New England Biolabs, Clontech and Gibco BRL. Other preferred plasmids and vectors can be found in: Glover, D. M. (1985), DNA cloning: a practical approach, Vol. I-III, IRL Press Ltd., Oxford; Rodriguez, R. L. and Denhardt, D. T. (eds) (1988), Vectors: a survey of molecular cloning vectors and their uses, 179-204, Butterworth, Stoneham; Goedeel, D. V. (1990), Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York. Plasmids which can be used to clone the gene constructs containing the nucleic acid sequences encoding the subunits into the host organism in a very preferred manner are: pUC18/19 (Roche Biochemicals), pKK-177-3H (Roche Biochemicals), pBTac2 (Roche Biochemicals), pKK223-3 (Amersham Pharmacia Biotech), pKK-233-3 (Stratagene) and pET (Novagen). Very particular preference is given to an expression system which is based on plasmids belonging to the pET series. Extreme preference is given to using plasmids of the same series for expressing both the nucleic acid sequence encoding the α subunit and the nucleic acid sequence encoding the β subunit.

In another embodiment, the present invention relates to a method for preparing nitrile hydratases. The method is characterized in that it is carried out using an expression system according to the invention as described above.

In a preferred embodiment, the method according to the invention is carried out such that the expression is performed at incubation temperatures of less than 30 degrees Celsius, preferably less than 25 degrees Celsius and very particularly preferably at ≦20 degrees Celsius. The embodiment in which alcohols, in particular ethanol, are added to the medium, during the expression, at a concentration of less than 10% (w/w), preferably less than 5% (w/w) and very particularly preferably 2-4% (w/w) is also advantageous. Implementing these measures results in insoluble proteins (inclusion bodies), which do not display any activity, either not being formed, or only being formed to a decreased extent, in the method according to the invention for preparing nitrile hydratases.

In another embodiment, the present invention relates to a host organism which possesses an expression system according to the invention. As already indicated above, the nucleic acid sequences encoding the nitrile hydratase subunits can, in a manner according to the invention as described above, be integrated into plasmids and transformed into host organisms. In addition, and as well as the expression system according to the invention, cloned genes for an amidase which may work stereoselectively (e.g. the amidase disclosed in W02004/005517 or EP 1318193) may also be present in the host organism. A whole-cell catalyst which has been prepared in this way is advantageously able to produce both the enzymes involved in the nitrile breakdown, thereby ensuring that the nitrile employed is immediately converted into the corresponding carboxylic acid. Whole-cell catalysts which comprise several enzymes which are involved in a reaction cascade have already been disclosed (EP1216304). They are used in the present invention in an equivalent manner. If rhamnose-inducible promoters are used, an organism as specified in DE10155928 should then be employed as the host organism or whole-cell catalyst. It is furthermore advantageous to use an E. coli BL21 codon plus which may have been modified in an equivalent manner to that described in DE10155928.

In order to match the expression of the enzymes with regard to their turnover rates, the respective nucleic acid sequences encoding the nitrile hydratase and the amidase can, in accordance with their turnover rates, be cloned in different plasmids having different copy numbers and/or promoters of differing strength in order to obtain differing strengths of expression of the nucleic acid sequences. In enzyme systems which are matched in this way, there is advantageously no accumulation of an intermediate compound, which may have an inhibitory effect, and the reaction under consideration can take place at an optimal overall rate. However, this is sufficiently well-known to the skilled person (Gellissen, G.; Piontek, M.; Dahlems, U.; Jenzelewski, V.; Gavagan, J. W.; DiCosimo, R.; Anton, D. L.; Janowicz, Z. A. (1996), Recombinant Hansenula polymorpha as a biocatalyst. Coexpression of the spinach glycolate oxidase (GO) and the S. cerevisiae catalase T (CTT1) gene, Appl. Microbiol. Biotechnol. 46, 46-54; Farwick, M.; London, M.; Dohmen, J.; Dahlems, U.; Gellissen, G.; Strasser, A. W.; DE19920712).

The skilled person is likewise familiar with preparing such whole-cell catalyst (Sambrook, J.; Fritsch, E. F. and Maniatis, T. (1989), Molecular cloning: a laboratory manual, 2nd ed., Cold Spring Harbor Laboratory Press, New York; Balbas, P. and Bolivar, F. (1990), Design and construction of expression plasmid vectors in E. coli, Methods Enzymol. 185, 14-37; Rodriguez, R. L. and Denhardt, D. T. (eds) (1988), Vectors: a survey of molecular cloning vectors and their uses, 205-225, Butterworth, Stoneham).

Another embodiment of the present invention relates to a method for preparing (amino)carboxylic acids or (amino)carboxamides which may, where appropriate, be enantiomer-enriched. This method is also carried out using a host organism or an expression system as described above. This means that the nitrile hydratases which are involved in the preparation of the carboxylic acids or carboxamides are obtained using a host organism as has just been described. For this application, the enzymes under consideration can be used in free form, as homogeneously purified compounds, or as recombinantly (rec-) prepared enzymes. Furthermore, the enzymes can also be used as components of an intact guest organism or in combination with the disrupted and, if desired, highly purified cell mass of the host organism.

It is likewise possible to use the enzymes in immobilized form (Sharma B. P.; Bailey L. F. and Messing R. A. (1982), Immobilisierte Biomaterialiern—Techniken und Anwendungen [Immobilized biomaterials—techniques and applications], Angew. Chem. 94, 836-852). The immobilization is advantageously effected by lyophilization (Paradkar, V. M.; Dordick, J. S. (1994), Aqueous-Like Activity of -Chymotrypsin Dissolved in Nearly Anhydrous Organic Solvents, J. Am. Chem. Soc. 116, 5009-5010; Mori, T.; Okahata, Y. (1997), A variety of lipid-coated glycoside hydrolases as effective glycosyl transfer catalysts in homogeneous organic solvents, Tetrahedron Lett. 38, 1971-1974; Otamiri, M.; Adlercreutz, P.; Matthiasson, B. (1992), Complex formation between chymotrypsin and ethyl cellulose as a means to solbilize the enzyme in active form in toluene, Biocatalysis 6, 291-305). Very particular preference is given to the lyophilization being carried out in the presence of surfactants such as Aerosol OT or polyvinylpyrrolidone or polyethylene glycol (PEG) or Brij 52 (diethylene glycol monocetyl ether) (Kamiya, N.; Okazaki, S.-Y.; Goto, M. (1997), Surfactant-horseradish peroxidase complex catalytically active in anhydrous benzene, Biotechnol. Tech. 11, 375-378).

Extreme preference is given to immobilizing on Eupergit®, in particular Eupergit C® and Eupergit 250L® (Röhm) (Eupergit.RTM. C, a carrier for immobilization of enzymes of industrial potential. Katchalski-Katzir, E.; Kraemer, D. M. Journal of Molecular Catalysis B: Enzymatic (2000), 10(1-3), 157-176).

Preference is also given to immobilizing on Ni-NTA in combination with the His tag (hexahistidine)-supplemented polypeptide (Purification of proteins using polyhistidine affinity tags. Bornhorst, Joshua A.; Falke, Joseph J. Methods in Enzymology (2000), 326, 245-254). The use as CLECs is likewise conceivable (St. Clair, N.; Wang, Y.-F.; Margolin, A. L. (2000), Cofactor-bound cross-linked enzyme crystals (CLEC) of alcohol dehydrogenase, Angew. Chem. Int. Ed. 39, 380-383).

Using these measures, it is possible to generate polypeptides (enzymes) which are stable, and can operate, in mixtures of aqueous and organic solvents, or in organic solvents on their own, from equivalent polypeptides which are rendered unstable by organic solvents.

The following strains are used in the experiments which are described below:

List of the strains employed. (Brandão PFB, Clapp JP and Bull AT (2002). Discrimination and taxonomy of geographically diverse strains of nitrile-metabolising actinomycetes using chemometric and molecular sequencing techniques. Environmental Microbiology 4, 262-276; see also PCT/EP04/00338).

TABLE 1

| | | α-subunit | β-subunit |
|---|---|---|---|
| Isolate | DSM | Seq. No. | Seq. No. |
| R. erythropolis | 15258 | 1 | 3 |
| 870-AN019 | | | |
| R. erythropolis | 15261 | 5 | 7 |
| ENG-AN033 | | | |
| R. erythropolis | 15265 | 9 | 11 |
| 871-AN042 | | | |
| R. rhodochrous | Russian National | 13 | 15 |
| M8 | Collection of | | |
| | Microorganisms | | |
| | VKPM-S-926 | | |

The following primers were used for the blunt-end cloning in pUC18/19 (xSmaI) (FIG. 1) of the nitrite hydratases and the p47K protein (Seq. ID No. 33) from the abovementioned strains:

| | | Amplified | Seq. |
|---|---|---|---|
| Primer | Primer sequence | subunits | No. |
| NH-Re-N | 5′-GCC CGC ATA AGA AAA | α, β, | 17 |
| | GGT GAA C | p47K | |
| NH-Re-C-p47K | 5′-GCA TGC CTT CAA ATC | α, β, | 18 |
| | AGC CTC | p47K | |

The first expression experiments in a one-vector expression system for nitrile hydratases from the R. erythropolis strains 870-AN019, 871-AN042 and ENG-AN033 were carried out using plasmids of the pUC18/19 series in different E. coli strains. In order to be able to identify the optimal expression host, the pUC18/19 constructs containing the nitrile hydratases from R. erythropolis strains 870-AN019, 871-AN042 and ENG-AN033 were transformed into different E. coli strains and expressed under the control of the lac promoter. The E. coli strains in this context were the following: JM109, DM5α, BL21 codon plus, BL21, HB101, MM294 and XL1blue.

The activities (units per g of dry cell mass) of the recombinant nitrile hydratases in the different hosts can be summarized as follows: it was found that the highest activity was obtained with E. coli BL21 codon plus RIL (from Stratagene) in which the copy numbers of the t-RNAs for arginine (AGA/AGG), isoleucine (AUA) and leucine (CUA), which t-RNAs are rare in the case of E. coli, are increased.

This host organism was constructed in order, in particular, to express genes having a codon usage which is adapted to a high GC content, for example the genes from rhodococci (72% GC).

The R. erythropolis 870-AN019 nitrile hydratase expressed in E. coli BL21 codon plus shows by far the highest activity, of 100 U/g of DBM, with this being followed by 870-AN019 nitrile hydratases expressed in DL5α (8 U/g of DBM) and ENG-AN033 nitrile hydratases expressed in BL21 codon plus (7 U/g of DBM).

The activities of all the other recombinant organisms were less than 2 U/g of DBM and were undetectable. An activity of 280 U/g of DBM was even achieved for the 870-AN019 nitrile hydratase under optimized conditions.

In that which follows, iron-dependent nitrile hydratases were expressed in a two-vector expression system, with α and β subunits each being present on a separate plasmid. The advantage of this system is that the two units are in each case directly under the control of the T7 promoter, which is employed, where appropriate, and the transcripts for the two genes are consequently formed to equal extents. The p47K helper protein (Seq. ID No. 33) was located downstream of one of the two subunits on a case-by-case basis. Stratagene plasmids of the pET series (pET22b and pET26b) were used as expression vectors. The aim was to express R. erythropolis 870-AN019 nitrile hydratase (Seq. Nos. 1 and 3).

The following primers were used for cloning the two subunits and the p47K protein (Seq ID No. 33):

| | | Amplified | Seq. |
|---|---|---|---|
| Primer | Primer sequence | orf | No. |
| NH019-α-for- | 5′-AGG GTC AAC CAT ATG | α | 19 |
| Nde | TCA GTA ACG | | |
| NH019-α-rev- | 5′-TGT CGG ATC CAT CAG | α | 20 |
| Bam | ACG GTG G | | |
| NH019-β-for- | 5′-AGC ACC ATA TGG ATG | β | 21 |
| Nde | GAG TAC AC | | |
| NH019-β-rev- | 5′-GTT GGG AAT TCA GGC | β | 22 |
| Eco | CGC AGG | | |
| NH019-p47K- | 5′-CGC GGA TCC AAG AAG | p47K | 23 |
| for-Bam | GAG ATA TAC ATG | | |
| NH019-p47K- | 5′-CCG GAA CGT TCA AAC | p47K | 24 |
| rev-Hind | GGT CTG G | | |

Primers which were derived from the R. erythropolis 870-AN019 nitrile hydratase sequence (Brandao P F B, Clapp J P, Bull A T (2003) Diversity of nitrile hydratase and amidase enzyme genes in Rhodococcus erythropoli recovered from geographically distinct habitats. Applied and Environmental Microbiology 69 (10): 5754-5766; see also PCT/EP04/00338), and which were provided with cleavage sites for the restriction endonucleases NdeI (N-terminally) and, respectively, BamHI (α subunit) or EcoRI (β subunit) C-terminally, were used for the α and β subunits. The primers for p47K were likewise derived from this organism and contained the cleavage sites for BamHI (N-terminally) and, respectively, HinDIII (C-terminally). The β subunit was cloned into pET26b while the α subunit and p47K were together cloned into pET22b. This resulted in the following plasmids, which were transformed into E. coli BL21 codon (+) RIL (see also FIGS. 2/3).

The activities of the R. erythropolis 870-AN019 nitrile hydratase in the E. coli strains BL21 and BL21 codon plus RIL (see above) were prepared. When using the above-described expression system, activities of approx. 1750 U/g of DBM and of 2750 U/g of DBM (based on benzonitrile as substrate) were achieved when using E. coli BL21 and E. coli BL21 codon (+), respectively. This signifies an increase by a factor of from 5.3 to 8.3 as compared with the one-vector expression systems.

However, it was true that 50% of the recombinant protein in the cell was present, in these experiments, as what are termed inclusion bodies. In order to further improve the activities of the nitrile hydratases, parameters such as IPTG concentration, temperature during the heterologous expression, and the supply of additives to the medium, were investigated. Reducing the IPTG concentration had no effect on the solubility of the recombinant nitrile hydratases. All the subsequent experiments were therefore carried out using 1 mM IPTG. Lowering the incubation temperature had the greatest effect in reducing the formation of inclusion bodies.

By reducing the temperature down to 20° C. after the heterologous expression had been induced, it was possible to transfer a large proportion of the enzyme into the soluble fraction. Adding 3% ethanol to the medium had a further positive effect. Under these conditions (1 mM IPTG, 20° C. incubation temperature, 3% added ethanol) it was possible to achieve an activity of 6480 U/g of DBM.

A further increase in activity was achieved by using a synthetic 870-AN019 nitrile hydratase gene. The corresponding nucleic acid sequences encoding the α and β subunits were prepared while taking into consideration the E. coli codon usage and retaining the amino acid sequences of the two genes. The intended advantage of these synthetic genes was that the DNA sequence would be optimally adapted for expression in E. coli and that consequently it would also be possible to dispense with using the “codon plus” strain. The subunits were then separately cloned into pET vectors, as previously described, and expressed in E. coli BL21 under the above optimized conditions. Using this strain, an activity of approx. 10 000 U/g of DBM was achieved. Consequently, this strain is more than twice as active as the wild-type R. erythropolis 870-AN019, which has an activity of approx. 4760 U/g of DBM when using benzonitrile as substrate. Table 2 provides a summary of the activities which were achieved.

In the E. coli host, the synthetic nitrile hydratase constitutes approx. 50% of the total cell protein formed, with approx. 20% being present as dissolved recombinant protein.

TABLE 2
Overview of the nitrile hydratase activities which
were measured using different expression systems.

| | 870-AN019 nitrile hydratase expression conditions | | |
|---|---|---|---|
| Expression | | | Synthetic |
| system | Native subunits, | Native subunits, | subunits |
| (Host & | standard medium, | medium + 3% | medium + 3% |
| promoter) | 26° C. | EtOH, 20° C. | EtOH, 20° C. |
| E. coli BL21 | 1750 U/g DBM | — | 10080 U/g DBM |
| (DE3) + T7 | | | |
| promoter | | | |
| E. coli BL21 | 2750 U/g DBM | 6480 U/g DBM | — |
| (DE3) codon | | | |
| usage & T7 | | | |
| promoter | | | |

In conclusion, the expression of cobalt-dependent nitrile hydratases was investigated in a two-vector expression system. The cobalt-dependent Rhodococcus rhodochrous M8 nitrile hydratase (Pogorelva TE, Ryabchenko LE, Sunzov NI, Yanenko AS (1996) Cobalt-dependent transcription of nitrile hydratase gene in Rhodococcus rhodochrous M8. FEMS Microbiology Letters 144: 191-195) was cloned and prepared using the above-described expression system according to the invention.

The nitrile hydratase sequence (Seq. ID Nos. 13 and 15) is deposited in GenBank (DDBJ, EMBL and NCBI DNA database) under the reference X86737 and is freely available. The following primers were used for cloning the individual subunits and the R. rhodochrous M8 p12K protein (Seq. ID No. 31):

| | | Amplified | Seq. |
|---|---|---|---|
| Primer | Primer sequence | subunits | No. |
| M8-α-for-Nde | 5′-AGG AAT ACG CAT ATG | α | 25 |
| | AGC GAGC ACG TC | | |
| M8-α-rev-Bam | 5′-GTG TGG ATC CAC TCA | α | 26 |
| | TAC GAT CAC TTC CTG | | |
| M8-β-for-Nde | 5′-AGG AAT GAG CAT ATG | β | 27 |
| | GAT GGT ATC CAC GAC A | | |
| M8-β-rev-Bam | 5′-ATC GGG ATC CTT TCA | β | 28 |
| | CGC AGA GAT CAG GTA | | |
| | CGG | | |
| M8-p12K-for- | 5′-CTC AGG ATC CAA GGA | p12K | 29 |
| Bam | GTG ATC GTA TGA GTG | | |
| | AAG AC | | |
| M8-p12K-rev- | 5′-ACA GGA GCT CTC AGT | p12K | 30 |
| Sac | CGA TGA TGG CC | | |

Primers which were derived from the M8 nitrile hydratase sequence and which were provided with cleavage sites for the restriction endonucleases NdeI (N-terminally) and, respectively, BamHI (C-terminally) were used for the α and β subunits. The primers for p12K were derived from the R. rhodochrous J1 sequence and contained the cleavage sites for BamHI (N-terminally) and, respectively, SacI (C-terminally). The β subunit (Seq. ID No. 15) was cloned into pET26b while the α subunit (Seq. ID No. 13) and p12K (Seq. ID No. 31) were cloned into pET22b. This resulted in the following plasmids (FIGS. 4/5), which were transformed into E. coli BL21 codon(+) RIL.

In contrast to the expression of iron-dependent nitrile hydratases in E. Coli, the cells had, in the case of the cobalt-dependent nitrile hydratase, to be precultured over several generations in order to accustom to the toxic cobalt (0.5 mM employed).

Table 3 compares the activities of the recombinant cobalt-dependent (R. rhodochrous M8) and iron-dependent (R. erythropoli 870-AN019) nitrile hydratases.

TABLE 3
Activities of the cobalt-dependent and iron-dependent nitrile
hydratases which were measured, following expression
in accordance with the invention, using acrylamide as substrate.

| Recombinant nitrile | Activity (U/mg); |
|---|---|
| hydratase from | substrate, acrylamide |
| R. rhodochrous M8 (Co) | 160 |
| R. erythropolis 870-AN019 | 250 |
| (Fe) (synthetic, see above) | |

It was thus possible to demonstrate that the expression system according to the invention can advantageously be used both for cobalt-dependent and for iron-dependent nitrile hydratases. It is possible, in particular, to achieve dramatic increases in activity when the expression systems and/or sequences are appropriately optimized. In addition, as a result of the successful separate expression of the nitrile hydratase subunits, it is possible to investigate novel combinations of subunits belonging to different nitrile hydratases, with this helping to increase the diversification of the nitrile hydratases and consequently the diversification of their properties. At the time of the invention, it was not possible to deduce this, in an obvious manner, from the prior art.

Within the context of the invention, “optically enriched (enantiomerically enriched, enantiomer-enriched) compounds” are understood as meaning the presence of one optical antipode in a mixture with the other at a concentration of >50 mol %.

The term “nucleic acid sequences” subsumes all types of single-stranded or double-stranded DNA as well as RNA, or mixtures thereof.

The organisms 870-AN019, ENG-AN033 and 871-AN042 were deposited by the applicant, on Oct. 22, 2002, in the Deutsche Sammlung für Mikroorganismen und Zellculkuren [German collection of microorganisms and cell cultures], Mascheroder Weg 4, 38124 Braunschweig in accordance with the Budapest treaty. The organism Rhodococcus rodochrous M8 is deposited in the all-Russian national collection of microorganisms under number VKPM-S-926. The corresponding sequences are contained in the gene database (see above).

According to the invention, the term “expression system” is understood such that it means nucleic acid-based biological material which is able, in organisms, to bring about the expression of the nucleic acid sequences which are inherent to it and which encode the nitrile hydratase subunits. This biological material comprises, in particular, plasmids and vectors.

The expressions “protein” and “polypeptide” are used synonymously within the context of the invention.

### DESCRIPTION OF THE FIGURES

FIG. 1: The vector map shows the general disposition of the nitrile hydratase α and β subunits and, respectively, the p47K orf in the pUC18 plasmid in an expression system using one vector.

FIGS. 2/3: The vector maps show the disposition of the nitrile hydratase α and β subunits and, respectively, the p47K orf from R. erythropolis 870-AN019 in plasmids of the pET series in an expression system using two vectors.

FIGS. 4/5: The vector maps show the disposition of the α and β subunits and, respectively, the p12K orf from R. rhodochrous M8 in plasmids of the pET series in an expression system using two vectors.

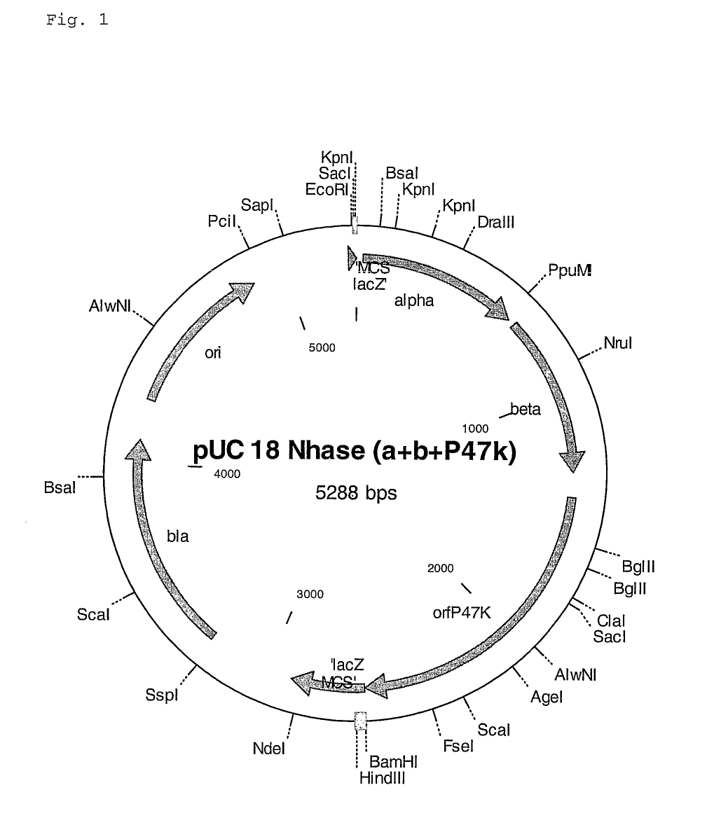

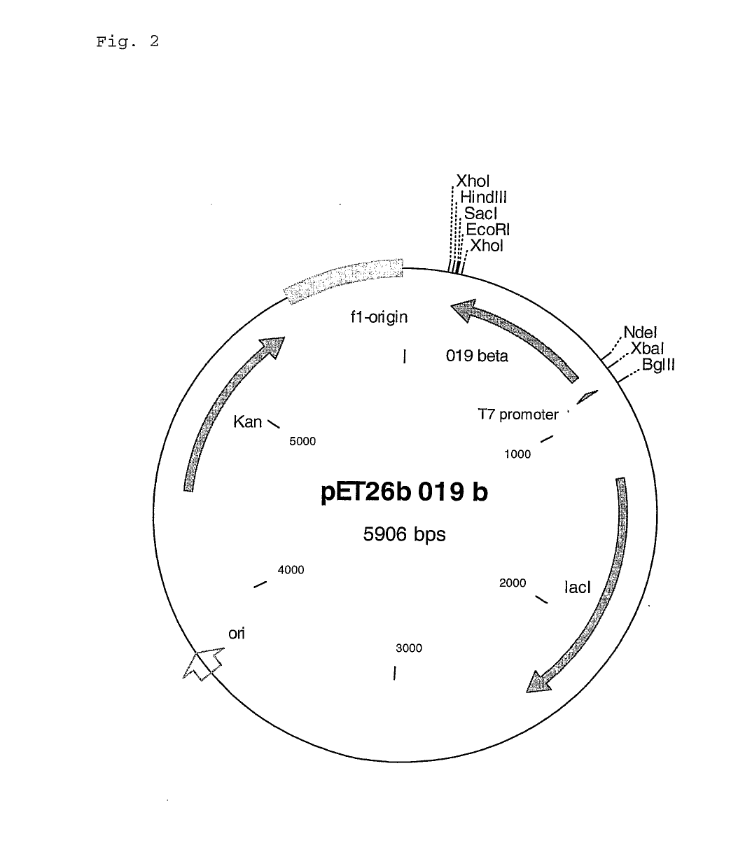

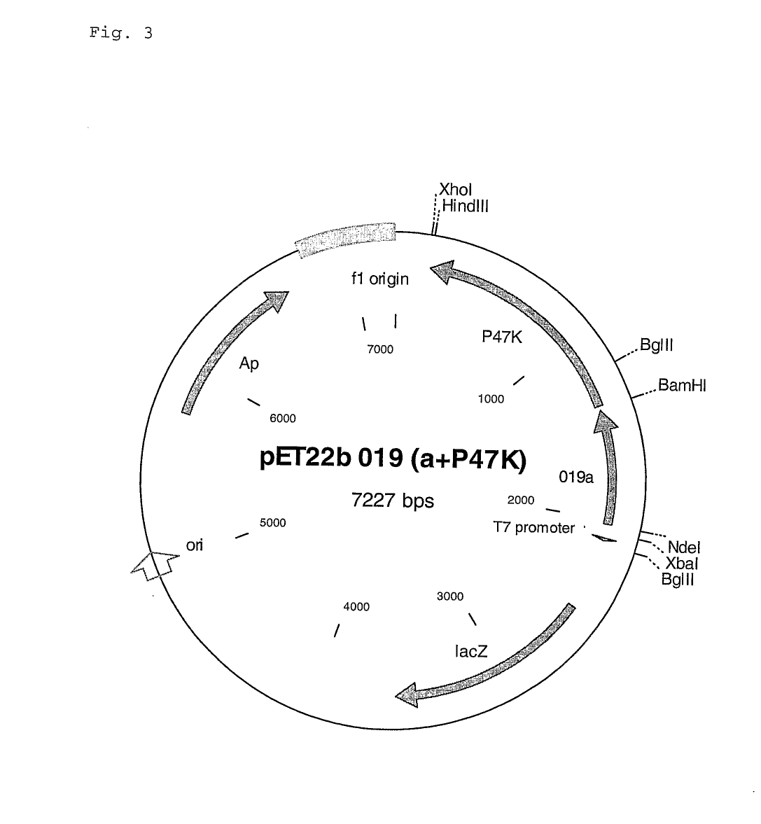

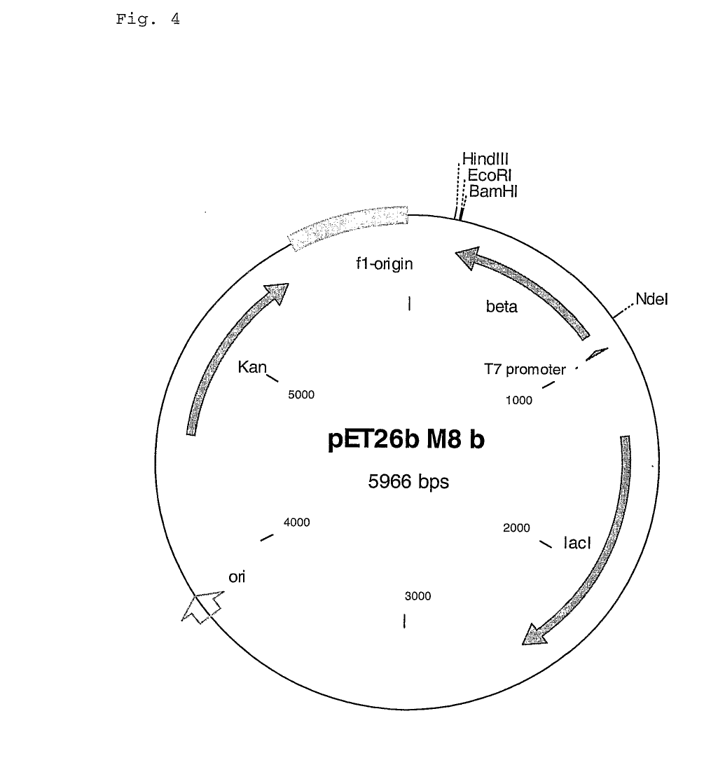

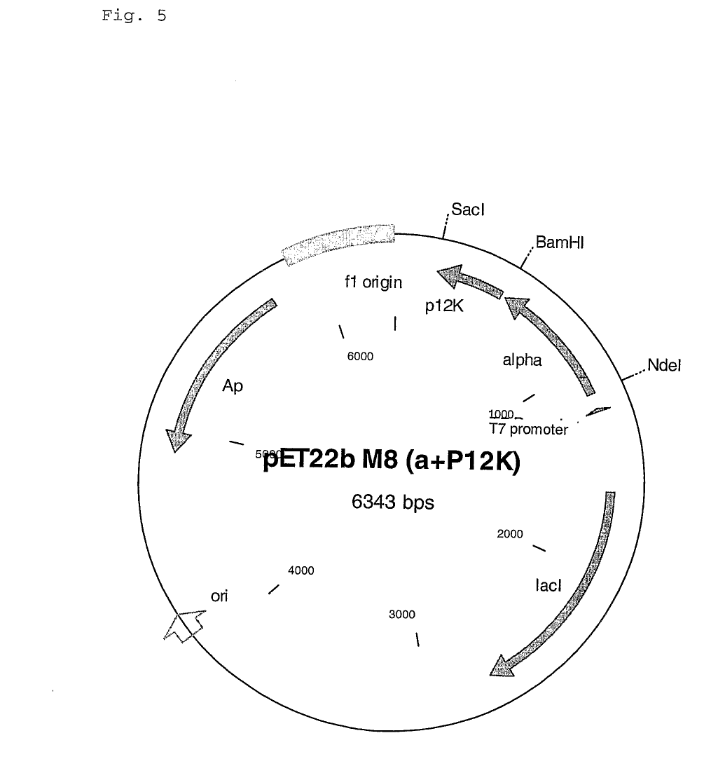

### EXPERIMENTAL SECTION

### Culturing Microorganisms

E. coli cells are cultured and stored as described in Sambrook et al. (Sambrook, J.; Fritsch, E. F. and Maniatis, T. (1989), Molecular cloning: a laboratory manual, 2nd ed., Cold Spring Harbor Laboratory Press, New York).

A chelate mineral medium (CMM) as described by Heald et al. 2001 (Heald S. C., P. F. B. Brandao, R. Hardicre and A. T. Bull, 2001: Physiology, biochemistry and taxonomy of deep-sea nitrile metabolising Rhodococcus strains. Antonie van Leeuwenhoek 80, 169-183) was used for culturing rhodococci. The CM medium is supplemented with 5 g of glucose/1. 2.4 mg of CoCl₂* 6 H₂O/l are used for strains containing cobalt-dependent enzymes while 50 mg of FeSO₄* 7 H₂0/l are used for strains containing iron-dependent enzymes.

The samples removed from the culture were diluted with potassium phosphate buffer used for the culturing (6 ml of a 200 g/l K₂HPO₄ solution/l and 4 ml of a 157.5 g/l KH₂PO₄ solution/l) such that the measurement range was between 0.05 and 0.3. The buffer served as the reference. Measurements were made at 600 nm.

PCR

For amplifying DNA fragments from rhodococci, the following components were pipetted in per 50 μl assay:
100 ng of chromosomal DNA
1 μl of dNTP mix (in each case 10 mM)
5 μl of buffer
2 μl of DMSO
0.5 μl of herculase (2.5 U)
H₂O to 50 μl

The thermocycler was programmed as follows:

A. 3 min 98° C.

B. 40 sec 98° C.

C. 40 X° C.

D. Y min 72° C.

E. 5 min 72° C.

F. 4° C.

Steps B, C and D were repeated 30 times. The annealing temperature X (C) is calculated from the melting temperature of the primers employed and the period of incubation Y (D) is calculated from the length of the gene to be amplified (rule, 1 kb of DNA equals 1 minute).

Digesting with Restriction Enzymes

The DNA to be cut is provided with 5 U of restriction enzyme and the appurtenant buffer and, if nothing else is required, incubated at 37 C. Chromosomal DNA is digested using 10 U of enzyme. The incubation period is from 1.5 to 2.5 hours.

Treating with Alkaline Phosphatase

In order to prevent vectors which have been cut with only one restriction endonuclease from religating with themselves, the protruding phosphate radical at the 5′ end is removed with alkaline phosphatase. Circular DNA can only be reformed by inserting a DNA fragment.

The vector which has been cut with a restriction endonuclease is incubated at 65° C. for 15 min in order to stop the restriction endonuclease. The dephosphorylation buffer is then added and the mixture is incubated, at 37° C. for 10 min, with 1 U of shrimp alkaline phosphatase. The enzyme is separated off by subsequently subjecting the vector DNA to gel electrophoresis.

Treating with T4 DNA Ligase

Vector and insert are used for the ligation in a ratio of 1:3. The volume is kept as low as possible (7-20 μl). The mixture is incubated overnight at 16° C. in ligation buffer and in the presence of 1 U of ligase.

Transforming

100 μl of competent cells are pipetted in to the ligation mixture and the whole is mixed by repeatedly drawing up into the pipette. After a 30 min incubation on ice, a heat-shock step is carried out at 42° C. for 45 sec and the mixture is incubated on ice for a further 2 min. 120-900 μl of SOC medium are added and the mixture is incubated at 37° C. for 45 min while being shaken. The mixture is subsequently plated out and incubated overnight at 37° C.

Expressing the different nitrile hydratases (table 1) in a one-vector expression system

The following protocol was used for the expression: 50 ml of LBₐₘₚ₁₀₀ medium containing 2 mM Fe citrate were inoculated to a concentration of 1% with an overnight culture. After an OD₆₀₀ of approx. 0.5 had been reached, expression of the nitrile hydratases in the different E. coli strains was induced with 1 mM IPTG (isopropylthiogalactoside). The cells were harvested approx. 24 hours after induction. The nitrile hydratases were expressed constitutively in strain DH5α since this strain does not overexpress the lac repressor. As a result, the step of inducing with IPTG is dispensed with in the case of this strain.

Activity Using Benzonitrile as Substrate:

The biotransformation was carried out on a 10 ml scale using approx. 100 mg of moist biomass (OD₆₀₀=5) in the potassium phosphate buffer (100 mM) pH 7.0. The incubation took place at 30° C. concentration was approx. 5 mM benzonitrile. Samples were removed every 5-10 min over a period of at most 1 hour. The sample volume was 100 μl and the reaction was stopped by adding 10 μl of 50% phosphoric acid.

The concentrations of benzonitrile and benzamide were then determined by means of HPLC:

Column: RP18 Phenomenex Hypersil ODS 5μ column (with precolumn)

Mobile phase: 10 mM K2HPO4 (pH 2.3)

Flow rate: 1 ml/min

Wavelength: 202 nm

Injection volume: 20 μl

Duration of HPLC run: 12-15 min

The activity was calculated by calculating a μmol turnover after one minute, which corresponds to one U (unit). Specific activities are given in U per g of DBM or mg of protein.

Expressing the R. erythropolis 870-AN019 nitrile hydratase in a two-vector expression system

The constructs containing T7 promoters were expressed in accordance with the following protocol:

50 ml of LBₐₘₚ₁₀₀ medium containing 2 mM Fe citrate and in each case 50 μg of kanamycin and ampicillin/ml were inoculated to a concentration of 1% with an overnight culture. After an OD₆₀₀ of approx. 0.5 had been reached, expression of the nitrile hydratases was induced with 1 mM IPTG (isopropylthiogalactoside). The cells were harvested approx. 24 hours after inducing at 26° C.

Expressing R. rhodochrous M8 nitrile hydratase in a two-vector expression system

The constructs containing T7 promoters were expressed in accordance with the following protocol:

50 ml of LBₐₘₚ₁₀₀ medium containing 0.5 mM CoCl₂ and in each case 50 μg of kanamycin and ampicillin/ml were inoculated to a concentration of 1% with an overnight culture. After an OD₆₀₀ of approx. 0.5 had been reached, expression of the NHases was induced with 1 mM IPTG (isopropylthiogalactoside). 3% (w/v) ethanol was also added to the medium. The cells were harvested approx. 24 hours after inducing at 26° C.

Determining Activity

The biotransformation for determining activity using the recombinant R. rhodochrous M8 and R. erythropolis 870-AN019 nitrile hydratases in E. coli took place on a small scale. The biotransformation is carried out in a 1.5 ml Eppendorf cup at 20° C. An OD of 0.4 was used in the biotransformation mixture. 500 μl of a 4% solution of acrylonitrile in 10 mm potassium phosphate buffer, pH 7.5, and also the buffer, are preincubated at 20° C. The reaction is started by adding the cells. The sum of the buffer volume and the cell volume is 500 μl. Immediately after the whole has been mixed, 100 μl are removed and pipetted into 1.5 μl of initially introduced concentrated HCl. After mixing, the sample is centrifuged down at 13 000 rpm for 2 min in an Eppendorf centrifuge and 70 μl of the supernatant are stored at −20° C. for HPLC analysis. Samples were removed every 5-10 over a period of at most 2 hours.

Analyzing Acrylonitrile, Acrylamide and Acrylic Acid

Using the HPLC method which is described below, it is possible to rapidly analyze acrylonitrile, acrylamide and acrylic acid and to determine the concentrations of these substances:

Column: Synergi 4μ Hydro-RP with precolumn

Mobile phase: 0.1% H₃PO₄ in 10% acetonitrile, 90% H₂0

Flow rate: 0.5 ml/min

Wavelength: 202 nm

Injection volume: 5 μl

Period of HPLC run: 10 min, last peak after 5.5 min

The activity was calculated by calculating a μmol turnover after one minute, which corresponds to one U (unit). Specific activities are given in U per g of DBM or mg of protein.

1. An expression system for simultaneously expressing the nucleic acid sequences encoding the different subunits of a nitrile hydratase,
characterized in that
the expression system comprises in each case at least one plasmid containing at least one nucleic acid sequence encoding the respective subunit.

2. The expression system as claimed in claim 1,
characterized in that
it is present in E. coli as host.

3. The expression system as claimed in one or both of the preceding claims,
characterized in that
the expression of the nucleic acid sequences encoding the subunits is under the control of what is in each case the same promoter.

4. The expression system as claimed in claim 3,
characterized in that
the promoter is a T7 promoter.

5. The expression system as claimed in one or more of the preceding claims,
characterized in that
at least one nucleic acid sequence encoding the p47K protein or the p12K protein is present per plasmid set employed.

6. The expression system as claimed in one or more of the preceding claims,
characterized in that
the nucleic acid sequences encoding the nitrile hydratase subunits are derived from rhodococcus strains.

7. The expression system as claimed in one or more of the preceding claims,
characterized in that
the nucleic acid sequences encoding the nitrile hydratase subunits are used in a form in which they are modified in accordance with the E. coli codon usage.

8. The expression system as claimed in one or more of the preceding claims,
characterized in that
the plasmids employed are those of the PET series.

9. A method for preparing nitrile hydratases using an expression system as claimed in one or more of claims 1 to 8.

10. A host organism which exhibits an expression system as claimed in one or more of claims 1 to 8.

11. A method for preparing optionally enantiomerically enriched (amino)carboxylic acids or (amino)carboxamides using a host organism as claimed in claim 10 or an expression system as claimed in one or more of claims 1 to 8.


SEQUENCE LISTING (34 sequences)

SEQ ID NO: 1 - DNA, 624 bp, Rhodococcus erythropolis
  CDS (1)..(624)
atgtcagtaacgatcgaccacacaacggagaacgccgcaccggcccaggcgccggtctccgatcgcgcgtgggccctgttccgcgcactcgacggtaagggattggtacccgacggttacgtcgagggatggaagaagaccttcgaggaggacttcagtccaaggcgcggagcggaattggtcgcgcgggcgtggaccgaccccgatttccggcaactgcttctcaccgacggtaccgccgcggttgcccagtacggatatctgggcccccagggcgaatacatcgtggcagtcgaagacaccccgaccctcaagaacgtgatcgtgtgctcgctgtgttcatgcaccgcgtggcccattctcggcctgccccctacctggtacaagagtttcgaataccgtgcgcgagtggtgcgtgagccacggaaggttctcttcgagatgggaaccgagatcgcgtcggacgtcgagatccgcgtctacgacaccaccgccgaaactcgctacatggttctcccgcaacgtcccgcaggcaccgaaggctggagccaggaacagcttcaggagatcgtcaccaaggactgcctgatcggcgtcgcagtcccgcaggtccccaccgtctga

SEQ ID NO: 2 - protein, 207 aa, Rhodococcus erythropolis
MSVTIDHTTENAAPAQAPVSDRAWALFRALDGKGLVPDGYVEGWKKTFEEDFSPRRGAELVARAWTDPDFRQLLLTDGTAAVAQYGYLGPQGEYIVAVEDTPTLKNVIVCSLCSCTAWPILGLPPTWYKSFEYRARVVREPRKVLFEMGTEIASDVEIRVYDTTAETRYMVLPQRPAGTEGWSQEQLQEIVTKDCLIGVAVPQVPTV

SEQ ID NO: 3 - DNA, 639 bp, Rhodococcus erythropolis
  CDS (1)..(639)
atggatggagtacacgatcttgccggagttcaaggcttcggcaaagtcccgcataccgtcaacgccgacatcggccccaccttccacgccgagtgggaacacctgccgtacagcctgatgttcgccggtgtcgccgaactcggggcattcagcgtcgacgaagttcgatacgtcgtcgagcggatggaaccacgccactacatgatgaccccgtactacgagaggtacgtcatcggcgtcgcgacactgatggtcgaaaagggaatcctgacgcaggaagaactcgaaagccttgcagggggaccgttcccactgtcgcggcccagcgaatccgaagggcggccggcacccgtcgagacgaccaccttcgaaatcggtcagcgagtacgcgtgcgcgacgagtacgttccggggcatattcgaatgcctgcgtactgccgcggacgagtgggaaccatctctcatcggactaccgagaagtggccgtttcccgacgcaatcggccacgggcgcaacgacgccggcgaagaaccgacgtaccacgtgaagttcgacgccgaggaattgttcggtagcgacaccgacggcggcagcgtcgtagtcgaccttttcgagggttacctcgagcctgcggcctga

SEQ ID NO: 4 - protein, 212 aa, Rhodococcus erythropolis
MDGVHDLAGVQGFGKVPHTVNADIGPTFHAEWEHLPYSLMFAGVAELGAFSVDEVRYVVERMEPRHYMMTPYYERYVIGVATLMVEKGILTQEELESLAGGPFPLSRPSESEGRPAPVETTTFEIGQRVRVRDEYVPGHIRMPAYCRGRVGTISHRTTEKWPFPDAIGHGRNDAGEEPTYHVKFDAEELFGSDTDGGSVVVDLFEGYLEPAA

SEQ ID NO: 5 - DNA, 624 bp, Rhodococcus erythropolis
  CDS (1)..(624)
atgtcagtaacgatcgaccacacaacggagaacgccgcaccggcccaggcgccggtctccgatcgcgcgtgggccctgttccgcgcactcgacggtaagggattggtacccgacggttacgtcgaaggatggaagaaaaccttcgaggaggacttcagtccaaggcgcggagcggaattggtcgcgcgggcgtggaccgaccccgagttccggcagttgcttctcaccgacggtaccgccgcggttgcccagtacggatacctgggcccccagggcgagtacatcgtggcagtcgaagacaccccgaccctcaagaacgtgatcgtgtgctcgctgtgttcatgcaccgcgtggcccattctcggcctgccccctacctggtacaagagtttcgaataccgtgcgcgagtggtgcgtgagccacggaaggttctctccgagatgggaaccgagatcgcgtcggacgtcgagatccgcgtctacgacaccaccgccgaaactcgctacatggttctcccgcaacgtcccgcaggcaccgaaggctggagccaggaacaactgcaggaaatcgtcaccaaggactgcctgatcggcgtcgcagtcccgcaggtccccaccgtctga

SEQ ID NO: 6 - protein, 207 aa, Rhodococcus erythropolis
MSVTIDHTTENAAPAQAPVSDRAWALFRALDGKGLVPDGYVEGWKKTFEEDFSPRRGAELVARAWTDPEFRQLLLTDGTAAVAQYGYLGPQGEYIVAVEDTPTLKNVIVCSLCSCTAWPILGLPPTWYKSFEYRARVVREPRKVLSEMGTEIASDVEIRVYDTTAETRYMVLPQRPAGTEGWSQEQLQEIVTKDCLIGVAVPQVPTV

SEQ ID NO: 7 - DNA, 639 bp, Rhodococcus erythropolis
  CDS (1)..(639)
atggatggagtacacgatcttgccggagttcaaggcttcggcaaagtcccgcataccgtcaacgccgacatcggccccaccttccacgccgagtgggaacacctgccgtacagcctgatgttcgccggtgtcgccgaactcggggcattcagcgtcgacgaagttcgatacgtcgtcgagcggatggaaccacgccactacatgatgaccccgtactacgagaggtacgtcatcggcgtcgcgacactgatggtcgaaaagggaatcctgacgcaggatgaactcgaaagccttgcagggggaccgttcccactgtcgcggcccagcgaatccgaagggcgtccggcacccgtcgagacgaccaccttcgaaatcggtcagcgagtacgcgtgcgcgacgagtacgttccggggcatattcgaatgcctgcgtactgccgcggacgagtgggaaccatctctcatcggactaccgagaagtggccatttcccgacgcaatcggccacgggcgcaacgacgccggcgaagaaccgacgtaccacgtgaagttcgccgccgaggaattgttcggtagcgacaccgacggcggcagcgtcgtagtcgaccttttcgagggttacctcgagcctgcggcctga

SEQ ID NO: 8 - protein, 212 aa, Rhodococcus erythropolis
MDGVHDLAGVQGFGKVPHTVNADIGPTFHAEWEHLPYSLMFAGVAELGAFSVDEVRYVVERMEPRHYMMTPYYERYVIGVATLMVEKGILTQDELESLAGGPFPLSRPSESEGRPAPVETTTFEIGQRVRVRDEYVPGHIRMPAYCRGRVGTISHRTTEKWPFPDAIGHGRNDAGEEPTYHVKFAAEELFGSDTDGGSVVVDLFEGYLEPAA

SEQ ID NO: 9 - DNA, 624 bp, Rhodococcus erythropolis
  CDS (1)..(624)
atgtcagtaacgatcgaccacacaacggagaacgccgcaccggcccaggcgccggtctccgaccgggcgtgggccctgttccgcgcactcgacggtaagggattggtacccgacggttacgtcgagggatggaagaagaccttcgaggaggacttcagtccaaggcgcggagcggaattggtcgcgcgggcgtggaccgaccccgagttccggcagttgcttctcaccgacggtaccgccgcggttgcccagtacggatatctgggcccccagggcgagtacatcgtggcagtcgaagacaccccgaccctcaagaacgtgatcgtgtgctcgttgtgttcatgcaccgcgtggcccattctcggcctgccccctacctggtacaagagtttcgaataccgtgcgcgagtggtgcgtgagccacggaaggttctctccgagatgggaaccgagatcgcgtcggacgtcgagatccgcgtctacgacaccaccgccgaaactcgctacatggttctcccgcaacgtcccgcaggcaccgaaggctggagccaggaacagcttcaagagatcgtcaccaaggactgcctgatcggcgtcgcagtcccgcaggtccccaccgtctga

SEQ ID NO: 10 - protein, 207 aa, Rhodococcus erythropolis; identical to SEQ ID NO: 6

SEQ ID NO: 11 - DNA, 639 bp, Rhodococcus erythropolis
  CDS (1)..(639)
atggatggagtacacgatcttgccggagttcaaggcttcggcaaagtcccgcataccgtcaacgccgacatcggccccaccttccacgccgagtgggaacacctgccgtacagcctgatgttcgccggtgtcgccgaactcggggcattcagcgtcgacgaagttcgatacgtcgtcgagcggatggaaccacgccactacatgatgaccccgtactacgagaggtacgtcatcggcgtcgcgacactgatggtcgaaaagggaatcctgacgcaggaagaactcgaaagccttgcagggggaccgttcccactgtcgcggccaagcgaatccgaagggcgtccggcacccgtcgagacgaccaccttcgaagtcggtcagcgagtacgcgtgcgcgacgagtacgttccggggcatattcgaatgcctgcgtactgccgcggacgagtgggaaccatctctcatcggactaccgagaagtggccatttcccgacgcaatcggccacgggcgcaacgacgccggcgaagaaccgacgtaccacgtgaagttcgacgccgaggaattgttcggtagcgacaccgacggcggcagcgtcgtagtcgaccttttcgagggttacctcgagcctgcggcctga

SEQ ID NO: 12 - protein, 212 aa, Rhodococcus erythropolis
MDGVHDLAGVQGFGKVPHTVNADIGPTFHAEWEHLPYSLMFAGVAELGAFSVDEVRYVVERMEPRHYMMTPYYERYVIGVATLMVEKGILTQEELESLAGGPFPLSRPSESEGRPAPVETTTFEVGQRVRVRDEYVPGHIRMPAYCRGRVGTISHRTTEKWPFPDAIGHGRNDAGEEPTYHVKFDAEELFGSDTDGGSVVVDLFEGYLEPAA

SEQ ID NO: 13 - DNA, 612 bp, Rhodococcus erythropolis
  CDS (1)..(612)
gtgagcgagcacgtcaataagtacacggagtacgaggcacgtaccaaggcaatcgaaactttgctgtacgagcgagggctcatcacgcccgccgcggtcgaccgagtcgtttcgtactacgagaacgagatcggcccgatgggcggtgccaaggtcgtggcgaagtcctgggtggaccctgagtaccgcaagtggctcgaagaggacgcgacggccgcgatggcgtcattgggctatgccggtgagcaggcacaccaaatttcggcggtcttcaacgactcccaaacgcatcacgtggtggtgtgcactctgtgttcgtgctatccgtggccggtgcttggtctcccgcccgcctggtacaagagcatggagtaccggtcccgagtggtagcggaccctcgtggagtgctcaagcgcgatttcggtttcgacatccccgatgaggtggaggtcagggtttgggacagcagctccgaaatccgctacatcgtcatcccggaacggccggccggcaccgacggttggtccgaggacgagctggcgaagctggtgagtcgggactcgatgatcggtgtcagtaatgcgctcacaccccaggaagtgatcgtatga

SEQ ID NO: 14 - protein, 203 aa, Rhodococcus erythropolis
VSEHVNKYTEYEARTKAIETLLYERGLITPAAVDRVVSYYENEIGPMGGAKVVAKSWVDPEYRKWLEEDATAAMASLGYAGEQAHQISAVFNDSQTHHVVVCTLCSCYPWPVLGLPPAWYKSMEYRSRVVADPRGVLKRDFGFDIPDEVEVRVWDSSSEIRYIVIPERPAGTDGWSEDELAKLVSRDSMIGVSNALTPQEVIV

SEQ ID NO: 15 - DNA, 690 bp, Rhodococcus erythropolis
  CDS (1)..(690)
atggatggtatccacgacacaggcggcatgaccggatacggaccggtcccctatcagaaggacgagcccttcttccactacgagtgggagggtcggaccctgtcgattctgacctggatgcatctcaagggcatgtcgtggtgggacaagtcgcggttcttccgggagtcgatggggaacgaaaactacgtcaacgagattcgcaactcgtactacacccactggctgagtgcggcagaacgtatcctcgtcgccgacaagatcatcaccgaagaagagcgaaagcaccgtgtgcaggagatcctcgagggtcggtacacggacaggaacccgtcgcggaagttcgatccggccgagatcgagaaggcgatcgaacggcttcacgagccccactccctagcacttccaggagcggagccgagtttctccctcggtgacaaggtcaaagtgaagaatatgaacccgctgggacacacacggtgcccgaaatatgtgcggaacaagatcggggaaatcgtcacctcccacggctgccagatctatcccgagagcagctccgccggcctcggcgacgatccccgcccgctctacacggtcgcgttttccgcccaggaactgtggggcgacgacggaaacgggaaagacgtagtgtgcgtcgatctctgggaaccgtacctgatctctgcgtga

SEQ ID NO: 16 - protein, 229 aa, Rhodococcus erythropolis
MDGIHDTGGMTGYGPVPYQKDEPFFHYEWEGRTLSILTWMHLKGMSWWDKSRFFRESMGNENYVNEIRNSYYTHWLSAAERILVADKIITEEERKHRVQEILEGRYTDRNPSRKFDPAEIEKAIERLHEPHSLALPGAEPSFSLGDKVKVKNMNPLGHTRCPKYVRNKIGEIVTSHGCQIYPESSSAGLGDDPRPLYTVAFSAQELWGDDGNGKDVVCVDLWEPYLISA

SEQ ID NO: 17 - DNA, 22 bp, Artificial Sequence
  Primer
gcccgcataagaaaaggtgaac

SEQ ID NO: 18 - DNA, 21 bp, Artificial Sequence
  Primer
gcatgccttcaaatcagcctg

SEQ ID NO: 19 - DNA, 24 bp, Artificial Sequence
  Primer
agggtgaaccatatgtcagtaacg

SEQ ID NO: 20 - DNA, 22 bp, Artificial Sequence
  Primer
tgtcggatccatcagacggtgg

SEQ ID NO: 21 - DNA, 23 bp, Artificial Sequence
  Primer
agcaccatatggatggagtacac

SEQ ID NO: 22 - DNA, 21 bp, Artificial Sequence
  Primer
gttgggaattcaggccgcagg

SEQ ID NO: 23 - DNA, 27 bp, Artificial Sequence
  Primer
cgcggatccaagaaggagatatacatg

SEQ ID NO: 24 - DNA, 22 bp, Artificial Sequence
  Primer
ccgcaacgttcaaacggtctgg

SEQ ID NO: 25 - DNA, 27 bp, Artificial Sequence
  Primer
aggaatacgcatatgagcgagcacgtc

SEQ ID NO: 26 - DNA, 30 bp, Artificial Sequence
  Primer
gtgtggatccactcatacgatcacttcctg

SEQ ID NO: 27 - DNA, 31 bp, Artificial Sequence
  Primer
aggaatgagcatatggatggtatccacgaca

SEQ ID NO: 28 - DNA, 33 bp, Artificial Sequence
  Primer
atcgggatcctttcacgcagagatcaggtacgg

SEQ ID NO: 29 - DNA, 35 bp, Artificial Sequence
  Primer
ctcaggatccaaggagtgatcgtatgagtgaagac

SEQ ID NO: 30 - DNA, 26 bp, Artificial Sequence
  Primer
acaggagctctcagtcgatgatggcc

SEQ ID NO: 31 - DNA, 315 bp, Rhodococcus erythropolis
  CDS (1)..(315)
atgagtgaagacacactcactgatcggctcccggcgactgggaccgccgcaccgccccgcgacaatggcgagcttgtattcaccgagccttgggaagcaacggcattcggggtcgccatcgcgctttcggatcagaagtcgtacgaatgggagttcttccgacagcgtctcattcactccatcgctgaggccaacggttgcgaggcatactacgagagctggacaaaggcgctcgaggccagcgtggtcgactcggggctgatcagcgaagatgagatccgcgagcgcatggaatcgatggccatcatcgactga

SEQ ID NO: 32 - protein, 104 aa, Rhodococcus erythropolis
MSEDTLTDRLPATGTAAPPRDNGELVFTEPWEATAFGVAIALSDQKSYEWEFFRQRLIHSIAEANGCEAYYESWTKALEASVVDSGLISEDEIRERMESMAIID

SEQ ID NO: 33 - DNA, 1200 bp, Rhodococcus erythropolis
  CDS (1)..(1200)
atggtcgacacacgacttccggtcacggtgctgtcaggtttcctgggcgccgggaagacgacactactcaacgagatcctgcgaaatcgagagggtcggcgggtcgcggtgatcgtcaacgacatgagcgaaatcaacatcgacagtgcagaagtcgagcgtgagatctcgctcagtcgctccgaggagaaactggtcgagatgaccaacggctgcatctgctgcactctgcgagaggatcttctttccgagatcagcgccttggccgccgatggccgattcgactacctactcatcgaatcttcgggcatctccgaaccgcttcccgtcgcagagacgttcacattcatcgataccgacggccacgccctcgccgacgtcgcccgactcgacaccatggtcaccgtcgtcgacggccacagttttctgcgcgactacacggctgggggccgcgtcgaagccgatgccccggaagacgaacgagacatcgcggatctgcttgtcgatcagatcgaatttgccgacgtcatcctggtgagcaaggccgatctcgtctcgcaccagcacctggtcgaattgaccgcagtcctgcgctctttgaacgcatccgctgcgatagttccgatgacgctcggtcgcatcccactcgacacgattctcgacaccggtttgttctcgctcgaaaaggctgcacaggcccccggatggttacaagaactccaaggtgaacacatccccgaaaccgaagagtacggaatcagttcggtggtgtaccgcgagcgcgcacccttccacccccaacggctgcatgatttcctcagcagcgagtggaccaacggaaagttacttcgggccaagggctactactggaatgccggccggttcaccgagatcgggagtatttctcaggccggtcatctcattcgccacggatacgtcggccgttggtggaagtttctaccccgtgacgagtggccggccgacgattaccgtcgtgacggaatcctcgacaagtgggaagaacccgtcggagactgccgacaagaactcgtcttcatcggccaagccatcgacccgtctcgactgcaccgagaactcgacgcgtgtctactcaccacagccgagatcgaactcgggccagacgtgtggaccacctggagcgaccccctgggcgtcggctataccgaccagaccgtttga

SEQ ID NO: 34 - protein, 399 aa, Rhodococcus erythropolis
MVDTRLPVTVLSGFLGAGKTTLLNEILRNREGRRVAVIVNDMSEINIDSAEVEREISLSRSEEKLVEMTNGCICCTLREDLLSEISALAADGRFDYLLIESSGISEPLPVAETFTFIDTDGHALADVARLDTMVTVVDGHSFLRDYTAGGRVEADAPEDERDIADLLVDQIEFADVILVSKADLVSHQHLVELTAVLRSLNASAAIVPMTLGRIPLDTILDTGLFSLEKAAQAPGWLQELQGEHIPETEEYGISSVVYRERAPFHPQRLHDFLSSEWTNGKLLRAKGYYWNAGRFTEIGSISQAGHLIRHGYVGRWWKFLPRDEWPADDYRRDGILDKWEEPVGDCRQELVFIGQAIDPSRLHRELDACLLTTAEIELGPDVWTTWSDPLGVGYTDQTV